(19) United States
(12) Patent Application Publication
(10) Pub. No.: US 2024/0382592 A1
(43) Pub. Date: Nov. 21, 2024

(54) METHODS FOR THE TREATMENT OF ANAPLASTIC LARGE CELL LYMPHOMA

(71) Applicants: INSTITUT NATIONAL DE LA SANTÉ ET DE LA RECHERCHE MÉDICALE, Paris (FR); FONDATION IMAGINE, Paris (FR); INSTUTUT GUSTAVE ROUSSY, Villejuif (FR); UNIVERSITÉ PARIS CITE, Paris (FR); UNIVERSITÉ PARIS-SACLAY, Gif-Sur-Yvette (FR); UNIVERSITÉ TOULOUSE III - PAUL SABATIER, Toulouse (FR); ASSISTANCE PUBLIQUE - HÔPITAUX DE PARIS, Paris (FR); CENTRE HOSPTIALIER UNIVERSITAIRE DE TOULOUSE, Toulouse (FR)

(72) Inventors: Erika CAUSSE BRUNET, Paris (FR); Thomas MERCHER, Villejuif (FR); Loélia BABIN, Toulouse (FR); Elie ROBERT, Villejuif (FR); Alice DARCHEN, Paris (FR); Laurence LAMANT, Toulouse (FR); Fabienne MEGGETTO-PRADELLE, Toulouse (FR)

(21) Appl. No.: 18/285,585

(22) PCT Filed: Apr. 8, 2022
(86) PCT No.: PCT/EP2022/059497

§ 371 Date: Oct. 4, 2023

(30) Foreign Application Priority Data
Apr. 9, 2021 (EP) 21305467.9

(51) Int. Cl. A61K 39/00; C07K 14/725; C07K 16/28

(52) U.S. Cl. CPC A61K 39/464411; A61K 39/4611; A61K 39/4631; C07K 14/7051; C07K 16/2803; C07K 2317/73

(57) ABSTRACT

Anaplastic large cell lymphoma (ALCL) is a rare and aggressive peripheral T-cell lymphoma affects lymph nodes and extra-nodal sites with characteristic skin lesions. Approximatively half of the tumors express the NPM1-ALK fusion from the translocation t(2;5)(p23;q32). In the present study, the inventors identify ROR2 as progressively up regulated thought tumorigenesis. Patient samples show a significantly high ROR2 expression (transcriptomic data) as well as a strong ROR2 protein expression (IHC) with some tumors displaying a clear membrane signal. ROR2 mRNA expression level is also positively correlated to NPM-ALK expression level in tumor cells and is not expressed in normal T cells. In addition, ROR2 protein level is significantly increased in resistant cells to the ALK inhibitor, crizotinib, used in clinical trials for children with refractory tumors. This result opens the road to ROR2 specific therapies: ROR2 inhibitors, monoclonal antibodies therapies or even ROR2 specific CAR cells, including for ALCL ALK(+) resistant tumors.

### FIELD OF THE INVENTION:

The present invention is in the field of medicine, in particular oncology.

### BACKGROUND OF THE INVENTION:

Anaplastic large cell lymphoma (ALCL) is a rare and aggressive peripheral T-cell lymphoma affects lymph nodes and extra-nodal sites with characteristic skin lesions. This lymphoma is divided into two subtypes, based on the expression of “Anaplastic lymphoma kinase” (ALK). 80% of the ALK(+) tumors express the NPM1-ALK fusion from the translocation t(2;5)(p23;q32). Anthracycline-based chemotherapy, such as CHOP (cyclophosphamide, doxorubicin, vincristine and prednisone) or CHOP-like regimens, constitutes the first-line of treatment. It may only be combined with radiotherapy in stage I/II disease. Pediatric patients have distinct protocols similar to protocols used for B-cell lymphomas, with other drugs such methotrexate, etoposide, and cytarabine being used. High-dose chemotherapy followed by autologous stem cell transplantation can also be performed, usually in cases of relapse or as first line treatment in cases with an adverse prognosis. Antibody-drug conjugate therapy (brentuximab velotin) may be given when at least one chemotherapy regimen is unsuccessful. Although ALCL ALK(+) are relatively sensitive to chemotherapy with high response rates, event-free survival is still between 65-75% and approximately 30% of early relapse are observed. These relapses are always associated with a very poor prognosis. In case of resistance to chemotherapy, treatments targeting ALK tyrosine kinase may be used, such as crizotinib. Unfortunately, mechanisms of resistance to ALK inhibitors have also been observed.

The present invention provides for a method of treating ALK positive anaplastic large cell lymphoma.

### SUMMARY OF THE INVENTION:

The present invention is defined by the claims. In particular, the present invention relates to methods for the treatment of anaplastic large cell lymphoma.

### DETAILED DESCRIPTION OF THE INVENTION

In the present study, the inventors demonstrate high-efficacy transformation of primary human (mature) T-cells upon precise engineering of the t(2;5)(p23;q35) translocation by CRISPR/Cas9 system. Their data show that human T cell survival increases drastically upon NPM1-ALK translocation induction, up to several months in cytokine free medium while normal T cells die in a few weeks. Immunodeficient mice transplanted with the NPM1-ALK(+) cells developed systemic disease in few months following injections, with nodal and skin involvements strikingly resembling human disease features. Interestingly the inventors observe the tumor formation with various types of cells recapitulating both the immune phenotype diversity (including the presence of CD4+ and CD8+ tumor cells), and the histological pattern (large or small cells) of NPMI-ALK(+) ALCL patient cells. Transcriptomic signature of engineered translocated clones in vivo (tumors) perfectly recapitulate patient tumors. Using progression analysis (from mature T cells to in vivo tumors including pre-transformed in vitro clones), the inventors could identify ROR2 as progressively up regulated thought tumorigenesis. ROR2 is a transmembrane receptor expressed at high level during early development and at low level in adult tissue. This receptor has been implicated in Wnt signaling pathway. Patient samples show a significantly high ROR2 expression (transcriptomic data) as well as a strong ROR2 protein expression (IHC) with some tumors displaying a clear membrane signal. ROR2 mRNA expression level is also positively correlated to NPM-ALK expression level in tumor cells and is not expressed in normal T cells. In addition, ROR2 protein level is increased in resistant cells to the ALK inhibitor, crizotinib, used in clinical trials for children with refractory tumors. This result opens the road to ROR2 specific therapies (ROR2 inhibitors, monoclonal antibodies therapies and ROR2 specific CART cells) among other things for ALCL ALK(+) resistant tumors.

### Main Definitions

As used herein, the term “anaplastic large cell lymphoma” or “ALCL” has its general meaning in the art and refers to a rare and aggressive peripheral T-cell non-Hodgkin lymphoma, belonging to the group of CD30-positive lymphoproliferative disorders, which affects lymph nodes and extranodal sites. It is comprised of two sub-types, based on the expression of a protein called anaplastic lymphoma kinase (ALK): ALK positive (AKL+) and ALK negative (AKL−) ALCL. ALCL accounts for approximately 3% of adult non-Hodgkin lymphomas and 10% to 20% of childhood lymphomas. Its prevalence is unknown. The ALK positive subtype usually affects children and young adults. The ALK negative subtype is more commonly found in older patients over the age of 40. ALCL is characterized by peripheral, mediastinal, or abdominal lymph node involvement. It manifests with the development of painless and enlarged lymph nodes, especially in the neck or armpit(axillary lymph nodes). General symptoms include loss of appetite and fatigue as well as fever, weight loss, and night sweats (B symptoms).

Mediastinal involvement manifests as cough, dypsnea and/or edema. ALCL can also extend to extranodal sites such as the bones, bone marrow, subcutaneous tissue, lungs, spleen and liver. In the ALK positive sub-type, the anaplastic lymphoma receptor tyrosine kinase ALK gene (2p23) is overexpressed due to a t(2;5)(p23;q35) translocation in more than half of the tumors. In the ALK negative sub-type, there is at least 2 recurrent chromosomal rearrangements. The first involves the DUSP22-IRF4 locus on 6p25.3 (referred to as DUSP22 rearrangements), and is most commonly present as t(6;7)(p25.3;q32.3). The second recurrent rearrangement involving the TP63 gene was identified among a constellation of genetic abnormalities affecting p53-related genes in ALCLs and related T-cell lymphomas

As used herein, the term “anaplastic lymphoma kinase” or “ALK” has its general meaning in the art and refers to the ALK tyrosine kinase receptor. An exemplary amino acid sequence for ALK is shown as SEQ ID NO:1.

SEQ ID NO: 1 >sp|Q9UM73|ALK_HUMAN ALK tyrosine
kinase receptor OS = Homo sapiens OX = 9606
GN = ALK PE = 1 SV = 3
MGAIGLLWLLPLLLSTAAVGSGMGTGQRAGSPAAGPPLQPREPLSYSRL
QRKSLAVDFVVPSLFRVYARDLLLPPSSSELKAGRPEARGSLALDCAPL
LRLLGPAPGVSWTAGSPAPAEARTLSRVLKGGSVRKLRRAKQLVLELGE
EAILEGCVGPPGEAAVGLLQFNLSELFSWWIRQGEGRLRIRLMPEKKAS
EVGREGRLSAAIRASQPRLLFQIFGTGHSSLESPTNMPSPSPDYFTWNL
TWIMKDSFPFLSHRSRYGLECSFDFPCELEYSPPLHDLRNQSWSWRRIP
SEEASQMDLLDGPGAERSKEMPRGSFLLLNTSADSKHTILSPWMRSSSE
HCTLAVSVHRHLQPSGRYIAQLLPHNEAAREILLMPTPGKHGWTVLQGR
IGRPDNPFRVALEYISSGNRSLSAVDFFALKNCSEGTSPGSKMALQSSF
TCWNGTVLQLGQACDFHQDCAQGEDESQMCRKLPVGFYCNFEDGFCGWT
QGTLSPHTPQWQVRTLKDARFQDHQDHALLLSTTDVPASESATVTSATF
PAPIKSSPCELRMSWLIRGVLRGNVSLVLVENKTGKEQGRMVWHVAAYE
GLSLWQWMVLPLLDVSDRFWLQMVAWWGQGSRAIVAFDNISISLDCYLT
ISGEDKILQNTAPKSRNLFERNPNKELKPGENSPRQTPIFDPTVHWLFT
TCGASGPHGPTQAQCNNAYQNSNLSVEVGSEGPLKGIQIWKVPATDTYS
ISGYGAAGGKGGKNTMMRSHGVSVLGIFNLEKDDMLYILVGQQGEDACP
STNQLIQKVCIGENNVIEEEIRVNRSVHEWAGGGGGGGGATYVFKMKDG
VPVPLIIAAGGGGRAYGAKTDTFHPERLENNSSVLGLNGNSGAAGGGGG
WNDNTSLLWAGKSLQEGATGGHSCPQAMKKWGWETRGGFGGGGGGCSSG
GGGGGYIGGNAASNNDPEMDGEDGVSFISPLGILYTPALKVMEGHGEVN
IKHYLNCSHCEVDECHMDPESHKVICFCDHGTVLAEDGVSCIVSPTPEP
HLPLSLILSVVTSALVAALVLAFSGIMIVYRRKHQELQAMQMELQSPEY
KLSKLRTSTIMTDYNPNYCFAGKTSSISDLKEVPRKNITLIRGLGHGAF
GEVYEGQVSGMPNDPSPLQVAVKTLPEVCSEQDELDELMEALIISKENH
QNIVRCIGVSLQSLPRFILLELMAGGDLKSFLRETRPRPSQPSSLAMLD
LLHVARDIACGCQYLEENHFIHRDIAARNCLLTCPGPGRVAKIGDFGMA
RDIYRASYYRKGGCAMLPVKWMPPEAFMEGIFTSKTDTWSFGVLLWEIF
SLGYMPYPSKSNQEVLEFVTSGGRMDPPKNCPGPVYRIMTQCWQHQPED
RPNFAIILERIEYCTQDPDVINTALPIEYGPLVEEEEKVPVRPKDPEGV
PPLLVSQQAKREEERSPAAPPPLPTTSSGKAAKKPTAAEISVRVPRGPA
VEGGHVNMAFSQSNPPSELHKVHGSRNKPTSLWNPTYGSWFTEKPTKKN
NPIAKKEPHDRGNLGLEGSCTVPPNVATGRLPGASLLLEPSSLTANMKE
VPLFRLRHFPCGNVNYGYQQQGLPLEAATAPGAGHYEDTILKSKNSMNQ
PGP

As used herein, the term “chemotherapy” refers to use of chemotherapeutic agents to treat a subject. As used herein, the term “chemotherapeutic agent” refers to chemical compounds that are effective in inhibiting tumor growth.

As used herein the term “resistance to chemotherapy” refers to the innate and/or acquired ability of cancer cells to evade the effects of chemotherapeutics. Chemotherapy resistance occurs when cancers that have been responding to a therapy suddenly begin to grow. The cancer cells are resisting the effects of the chemotherapy.

As used herein the term “CHOP” refers to the acronym for a chemotherapy regimen used in the treatment of non-Hodgkin lymphoma and is being studied in the treatment of other types of cancer. CHOP consists of: Cyclophosphamide (an alkylating agent which damages DNA by binding to it and causing the formation of cross-links), Hydroxydaunorubicin also called doxorubicin or Adriamycin (an intercalating agent which damages DNA by inserting itself between DNA bases), Oncovin or vincristine (which prevents cells from duplicating by binding to the protein tubulin) and Prednisone or Prednisolone (which are corticosteroids).

As used herein, the term “anaplastic lymphoma kinase inhibitor” or “ALK inhibitor” is defined herein to refer to a compound or biologic agent which targets, decreases or inhibits the synthesis or biological activity of anaplastic lymphoma kinase (ALK).

As used herein the term “resistance to ALK inhibitors” is used in its broadest context to refer to the reduced effectiveness of at least one ALK inhibitor to inhibit the growth of a cell, kill a cell or inhibit one or more cellular functions, and to the ability of a cell to survive exposure to an agent designed to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced. Accordingly, the term “resistant” refers to the repeated outbreak of cancer, or a progression of cancer independently of whether the disease was cured before said outbreak or progression.

As used herein, the term “ROR2” has its general meaning in the art and refers to the tyrosine-protein kinase transmembrane receptor ROR2. The term is also known as neurotrophic tyrosine kinase, receptor-related 2 (NTRKR2). An exemplary amino acid sequence for ROR2 is shown as SEQ ID NO:2. In particular, the extracellular domain of ROR2 ranges from the amino acid residue at position 34 to the amino acid residue at position 403 in SEQ ID NO:2.

SEQ ID NO: 2 >sp|Q01974|ROR2_HUMAN Tyrosine-
protein kinase transmembrane receptor ROR2
OS = Homo sapiens OX = 9606 GN = ROR2 PE = 1
SV = 2
MARGSALPRRPLLCIPAVWAAAALLLSVSRTSGEVEVLDPNDPLGPLDG
QDGPIPTLKGYFLNFLEPVNNITIVQGQTAILHCKVAGNPPPNVRWLKN
DAPVVQEPRRIIIRKTEYGSRLRIQDLDTTDTGYYQCVATNGMKTITAT
GVLFVRLGPTHSPNHNFQDDYHEDGFCQPYRGIACARFIGNRTIYVDSL
QMQGEIENRITAAFTMIGTSTHLSDQCSQFAIPSFCHFVFPLCDARSRT
PKPRELCRDECEVLESDLCRQEYTIARSNPLILMRLQLPKCEALPMPES
PDAANCMRIGIPAERLGRYHQCYNGSGMDYRGTASTTKSGHQCQPWALQ
HPHSHHLSSTDFPELGGGHAYCRNPGGQMEGPWCFTQNKNVRMELCDVP
SCSPRDSSKMGILYILVPSIAIPLVIACLFFLVCMCRNKQKASASTPQR
RQLMASPSQDMEMPLINQHKQAKLKEISLSAVREMEELGEDRFGKVYKG
HLFGPAPGEQTQAVAIKTLKDKAEGPLREEFRHEAMLRARLQHPNVVCL
LGVVTKDQPLSMIFSYCSHGDLHEFLVMRSPHSDVGSTDDDRTVKSALE
PPDFVHLVAQIAAGMEYLSSHHVVHKDLATRNVLVYDKLNVKISDLGLF
REVYAADYYKLLGNSLLPIRWMAPEAIMYGKFSIDSDIWSYGVVLWEVF
SYGLQPYCGYSNQDVVEMIRNRQVLPCPDDCPAWVYALMIECWNEFPSR
RPRFKDIHSRLRAWGNLSNYNSSAQTSGASNTTQTSSLSTSPVSNVSNA
RYVGPKQKAPPFPQPQFIPMKGQIRPMVPPPQLYVPVNGYQPVPAYGAY
LPNFYPVQIPMQMAPQQVPPQMVPKPSSHHSGSGSTSTGYVTTAPSNTS
MADRAALLSEGADDTQNAPEDGAQSTVQEAEEEEEGSVPETELLGDCDT
LQVDEAQVQLEA

As used herein, the term “agent capable of inducing cell death of ROR2 expressing cancer cells” refers to any molecule that under cellular and/or physiological conditions is capable of inducing cell death of ROR2 expressing cancer cells. In particular, the agent is capable of inducing apoptosis of ROR2 expressing cancer cells. In some embodiments, the agent is capable of depleting ROR2 cancer cells. As used herein, the term “depletion” with respect to cancer cells, refers to a measurable decrease in the number of ROR2 expressing cancer cells in the patient. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of ROR2 cancer cells in the patient below detectable limits.

As used herein, the term “ROR2 inhibitor” refers to a molecule that partially or fully blocks, inhibits, or neutralizes a biological activity or expression of ROR2. A ROR2 inhibitor can be a molecule of any type that interferes with the signalling associated with ROR2 in a cell, for example, either by decreasing transcription or translation of ROR2-encoding nucleic acid, or by inhibiting or blocking ROR2 polypeptide activity, or both. Examples of ROR2 inhibitors include, but are not limited to, antisense polynucleotides, interfering RNAs, catalytic RNAs, RNA-DNA chimeras, ROR2-specific aptamers, anti-ROR2 antibodies, ROR2-binding fragments of anti-ROR2 antibodies, ROR2-binding small molecules, ROR2-binding peptides, and other polypeptides that specifically bind ROR2 (including, but not limited to, ROR2-binding fragments of one or more ROR2 ligands, optionally fused to one or more additional domains), such that the interaction between the ROR2 inhibitor and ROR2 results in a reduction or cessation of ROR2 activity or expression.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art(see Kabat et al., 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21 (11): 484-490; and WO 06/030220, WO 06/003388. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light(VL) and heavy (VH) chains determine binding recognition and specificity to the antigen. The constant region domains of the light(CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

As used herein, the term “fully human” refers to an immunoglobulin, such as an antibody or antibody fragment, where the whole molecule is of human origin or consists of an amino acid sequence identical to a human form of the antibody or immunoglobulin.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

As used herein, the term “bispecific antibody” has its general meaning in the art and refers to an artificial, hybrid antibody having two different pairs of heavy and light chain and also two different antigen-binding sites.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. Moreover, when expressed in T-cells, CARs advantageously do not dimerize with endogenous T cell receptor (TCR) alpha and beta chains. The chimeric antigen receptor the present invention typically comprises an extracellular hinge domain, a transmembrane domain, and an intracellular T cell signaling domain.

As used herein the term “CAR cell” refers to a cell that has been genetically engineered to express a CAR. In particular, the term encompasses CAR-T cells, CAR NK cells and CAR-MAIT cells. The definition of CAR T-cells encompasses all classes and subclasses of T-lymphocytes including CD4+, CD8+ T cells, gamma delta T cells as well as effector T cells, memory T cells, regulatory T cells, and the like. The cells that are genetically modified may be “derived” or “obtained” from the patient who will receive the treatment using the genetically modified cells or they may “derived” or “obtained” from a different patient.

As used herein, the term “T cell” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, and gamma delta T cells. As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8+ T cell” refers to the pool of CD8+ T cells of the patient that have left the blood stream and have migrated into a tumor. As used herein, the term “CD4+ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4+ T cells become activated when they are presented with peptide antigens by MHC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCR5 (Tfh) and as well as subtype-specific expression of cytokines and transcription factors including T-bet, GATA3,EOMES, RORγT, BCL6 and FoxP3. As used herein, the term “gamma delta T cell” has its general meaning in the art. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62:1-5) or by other intracellular parasites, such as mycobacteria (Constant et al., Infection and Immunity, December 1995, vol. 63, no. 12:4628-4633) or protozoa (Behr et al., Infection and Immunity, 1996, vol. 64, no. 8:2892-2896). They may also be cancer cells (Poccia et al., J. Immunol., 159:6009-6015; Fournie and Bonneville, Res. Immunol., 66th Forum in Immunology, 147:338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

As used herein, the term “NK cell” has its general meaning in the art and refers to a sub-population of lymphocytes that is involved in innate or non-conventional immunity. NK cells can be identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response (“NK cell activities”). Any subpopulation of NK cells will also be encompassed by the term NK cells.

As used herein, the term “MAIT cell” has its general meaning in the art and refers to a subset of nonconventional T cells. In humans, MAIT cells are found in the blood, liver, and mucosae, defending against microbial activity and infection. MAIT cells are characterized by a semi-invariant T cell receptor alpha (TCRα) chain (Vα 7.2-Jα 33/20/12 in humans), that can combine with a restricted number of possible TCRβ chains. This semi-invariant TCR is restricted by the monomorphic, highly conserved, MHC class-I related 1 (MR1) molecule. In contrast to conventional T cells that recognize classical MHC-peptide complexes, MAIT cells recognize microbial-derived riboflavin precursor derivatives such as 5-OP-RU or 5-OE-RU, presented by MR1. Adult MAIT cells are easily identified by flow cytometry as CD3+ CD4− Va7.2+ CD161high (or IL-18ahigh or CD26high) using the corresponding staining. Upon recognition of MR1 ligands, MAIT cells release inflammatory cytokines (IFNγ, TNFα, IL-17) and mediate perforin-dependent cytotoxicity of target cells. MAIT cells are preferentially localized in the liver and mucosae, including lung and intestine, and are also abundant in the adult peripheral blood (1-10% of T cells) while they are very few in cord blood (<0.1% of T cells).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the expression “enhanced therapeutic efficacy” relative to cancer refers to a slowing or diminution of the growth of cancer cells, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy. As used herein, the expression “relative to” when used in the context of comparing the activity and/or efficacy of a combination composition to the activity and/or efficacy of one compound alone, refers to a comparison using amounts known to be comparable according to one of skill in the art.

As used herein the term “co-administering” as used herein means a process whereby the combination of the agent capable of inducing cell death of ROR2 expressing cancer cells and the ALK inhibitor, is administered to the same patient. The agent capable of inducing cell death of ROR2 expressing cancer cells and the ALK inhibitor may be administered simultaneously, at essentially the same time, or sequentially. If administration takes place sequentially, the agent capable of inducing cell death of ROR2 expressing cancer cells is administered before the ALK inhibitor. The agent capable of inducing cell death of ROR2 expressing cancer cells and the ALK inhibitor need not be administered by means of the same vehicle. The agent capable of inducing cell death of ROR2 expressing cancer cells and the ALK inhibitor may be administered one or more times and the number of administrations of each component of the combination may be the same or different. In addition, the agent capable of inducing cell death of ROR2 expressing cancer cells and the ALK inhibitor need not be administered at the same site.

As used the terms “combination” and “combination therapy” are interchangeable and refer to treatments comprising the administration of at least two compounds administered simultaneously, separately or sequentially. As used herein the term “co-administering” as used herein means a process whereby the combination of at least two compounds is administered to the same patient. The at least two compounds may be administered simultaneously, at essentially the same time, or sequentially. The at least two compounds can be administered separately by means of different vehicles or composition. The at least two compounds can also administered in the same vehicle or composition (e.g. pharmaceutical composition). The at least two compounds may be administered one or more times and the number of administrations of each component of the combination may be the same or different.

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects.

The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of a inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a inhibitor of the present invention may also be administered using a weekly, biweekly or triweekly dosing period. The dosing period may be restricted to, e.g., 8weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

### Methods of the Present Invention

Accordingly, the first object of the present invention relates to a method of treating an anaplastic large cell lymphoma in patient in need thereof comprising administering to the patient a therapeutically effective amount of an agent capable of inducing cell death of ROR2 expressing cancer cells.

A further object of the present invention relates to a method of treating an anaplastic large cell lymphoma resistant to chemotherapy in patient in need thereof comprising administering to the patient a therapeutically effective amount of an agent capable of inducing cell death of ROR2 expressing cancer cells.

In some embodiments, the anaplastic large cell lymphoma is ALK positive.

In some embodiments, the anaplastic large cell lymphoma is ALK negative.

A further object of the present invention relates to a method of treating a ALK positive anaplastic large cell lymphoma in a patient in need thereof comprising administering to the patient a therapeutically effective combination comprising at least one ALK inhibitor and an agent capable of inducing cell death of ROR2 expressing cancer cells.

A further object of the present invention relates to method of treating a ALK positive anaplastic large cell lymphoma resistant to chemotherapy in a patient in need thereof comprising administering to the patient a therapeutically effective combination comprising at least one ALK inhibitor and an agent capable of inducing cell death of ROR2 expressing cancer cells.

A further object of the present invention relates to a method of treating a ALK positive anaplastic large cell lymphoma resistant to ALK inhibitors in a patient in need thereof comprising administering to the patient a therapeutically effective amount of an agent capable of inducing cell death of ROR2 expressing cancer cells.

A further object of the present invention relates to a method for enhancing the potency of a ALK inhibitor administered to a patient suffering from a ALK positive anaplastic large cell lymphoma as part of a treatment regimen, the method comprising administering to the patient a pharmaceutically effective amount of an agent capable of inducing cell death of ROR2expressing cancer cells in combination with at least one ALK inhibitor.

A further object of the present invention relates to method of preventing resistance to chemotherapy in a patient suffering from a cancer comprising administering to the patient a therapeutically effective amount of an agent capable of inducing cell death of ROR2 expressing cancer cells.

A further object of the present invention relates to a method of preventing resistance to an administered ALK inhibitor in a patient suffering from a cancer comprising administering to the patient a therapeutically effective amount of an agent capable of inducing cell death of ROR2 expressing cancer cells.

### Chemotherapeutic Agents

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone;

aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®;

razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4 (5)-imidazoles, 4-hydroxytamoxifen, trioxifene, keoxifene,

LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the chemotherapy is a combination of Cyclophosphamide, doxorubicin, vincristine (Oncovin), and prednisolone (CHOP).

### ALK Inhibitors

Suitable ALK inhibitors for use in the present invention also include (a) Crizotinib (also known as PF02341066 and sold under the tradename XALKORI® by Pfizer), (b) Alectinib (also known as CH5424802) described in PCT Application WO WO2010/143664 (Chugai), which is hereby incorporated by reference in its entirety; (c) 5-Chloro-N4-[2-(isopropylsulfonyl) phenyl]-N2-[2-methoxy-4-[4-(4-methylpiperazin-1-yl) piperidin-1-yl] phenyl] pyrimidine-2,4-diamine (also known as TAE684) described in PCT Application WO WO2005/016894 (Novartis), which is hereby incorporated by reference in its entirety; (d) CEP28122 having the chemical name (1S,2S,3R,4R)-3-[(5-Chloro-2-[[(7S)-1-methoxy-7-(morpholin-4-yl)-6,7,8,9-tetrahydro-5H-benzocyclohepten-2-yl]amino]pyrimidin-4-yl)amino]bicyclo[2.2.1]hept-5-ene-2-carboxamide mesylate hydrochloride described in PCT Application WO WO2008/051547 (Cephalon), which is hereby incorporated by reference in its entirety; and (e)(f) Lorlatinib (also known as PF06463922) described in PCT application WO2013132376 and having the chemical name (16R)-19-amino-13-fluoro-4,8,16-trimethyl-9-oxo-17-oxa-4,5,8,20-tetraazatetracyclo[16.3.1.0\hat{ }{2,6}.0\hat{ }{10,15}]docosa-1(22),2,5,10(15),11,13,18,20-octaene-3-carbonitrile; or any pharmaceutically acceptable salt thereof.

### ROR2 Inhibitors

In some embodiments, the agent of inducing cell death of ROR2 expressing cancer cells is a ROR2 inhibitor.

In some embodiments, the ROR2 inhibitor is an inhibitor of ROR2 expression. An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of ROR2 mRNA by binding thereto and thus preventing protein translation or increasing mRNA degradation, thus decreasing the level of ROR2, and thus activity, in a cell. For example, antisense oligonucleotides of at least about 15 bases and complementary to unique regions of the mRNA transcript sequence encoding ROR2 can be synthesized, e.g., by conventional phosphodiester techniques. Methods for using antisense techniques for specifically inhibiting gene expression of genes whose sequence is known are well known in the art(e.g. see U.S. Pat. Nos. 6,566,135; 6,566,131; 6,365,354; 6,410,323; 6,107,091;6,046,321; and 5,981,732). Small inhibitory RNAs (siRNAs) can also function as inhibitors of expression for use in the present invention. ROR2 gene expression can be reduced by contacting a patient or cell with a small double stranded RNA (dsRNA), or a vector or construct causing the production of a small double stranded RNA, such that ROR2 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing ROR2. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the error prone non homologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the agent is a small organic molecule which inhibits the kinase activity of ROR2 or all molecule that can inhibit ROR2 function.

### ROR2 Antibodies

In some embodiments, the agent is an antibody having binding affinity for ROR2. In some embodiments, the agent is an antibody directed against the extracellular domain of ROR2. In some embodiments, the antibody leads to the inhibition of ROR2 activity. In some embodiments, the antibody leads to the internalisation of ROR2 in cancer cells. In some embodiments, the antibody leads to the depletion of ROR2 expression cancer cells (e.g. an antibody-drug conjugate as described herein after).

In some embodiments, the antibody is a humanized antibody or a chimeric antibody.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

### ROR2 Depleting Antibodies

In some embodiments, the antibody suitable for depletion of ROR2 cancer cells mediates antibody-dependent cell-mediated cytotoxicity.

As used herein the term “antibody-dependent cell-mediated cytotoxicity” or “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein, the term “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of cancer cells is a full-length antibody. In some embodiments, the full-length antibody is an IgG1 antibody. In some embodiments, the full-length antibody is an IgG3 antibody.

In some embodiments, the antibody suitable for depletion of cancer cells comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of cancer cells is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1176195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17:176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B1).

In some embodiments, the antibody suitable for depletion of cancer cells mediated complement dependant cytotoxicity.

As used herein, the term “complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of cancer cells mediates antibody-dependent phagocytosis.

As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

### ROR2 Multispecific Antibodies

In some embodiments, the antibody suitable for depletion of ROR2 cancer cells is a multispecific antibody comprising a first antigen binding site directed against ROR2 and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the multispecific antibody is thus a bispecific antibody. In some embodiments, the bispecific antibody is a BiTE. As used herein, the term “Bispecific T-cell engager” or “BiTE” refers to a bispecific antibody that is a recombinant protein construct composed of two flexibly connected single-chain antibodies (scFv). One of said scFv antibodies binds specifically to a selected, target cell-expressed tumour antigen (i.e. ROR2), the second binds specifically to another molecule such as CD3, a subunit of the T-cell receptor complex on T cells. In some embodiments, the BiTE antibodies are capable of binding T cells transiently to target cells and, at the same time, activating the cytolytic activity of the T cells. The BiTE-mediated activation of the T cells requires neither specific T-cell receptors on the T cells, nor MHC I molecules, peptide antigens or co-stimulatory molecules on the target cell.

### ROR2 Antibody-Drug Conjugates

In some embodiments, the antibody suitable for depletion of cancer cells is conjugated to a therapeutic moiety, i.e. a drug.

In some embodiments, the therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of cancer cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of cancer cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45 (12): 3580-3584) and have anti-cancer (U.S. Pat. No. 5,663,149) and antifungal activity (Pettit et al., (1998) Antimicrob. Agents and Chemother. 42:2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Patent Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of cancer cells is conjugated to pyrrolo [2,1-c] [1,4]-benzodiazepine (PDB) or an analog, derivative or prodrug thereof. Suitable PDBs and PDB derivatives, and related technologies are described in, e.g., Hartley J. A. et al., Cancer Res 2010; 70 (17): 6849-6858; Antonow D. et al., Cancer J 2008; 14 (3): 154-169; Howard P. W. et al., Bioorg Med Chem Lett 2009; 19:6463-6466 and Sagnou et al., Bioorg Med Chem Lett 2000; 10 (18): 2083-2086.

In some embodiments, the antibody suitable for depletion of cancer cells is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of cancer cells is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo [2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of cancer cells is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art(See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent(e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

### ROR2 CAR-Cells

In some embodiments, the agent is a CAR-cell wherein the CAR comprises at least an extracellular antigen binding domain specific for ROR2.

In some embodiments, the CAR-cell is a CAR-T cell, a CAR-NK cell or a CAR-MAIT cell.

In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28.

In some embodiments, the CAR comprises a chimeric fusion protein comprising an extracellular antigen binding domain specific for ROR2, a transmembrane domain and an intracellular signaling domain comprising a functional signaling domain derived from a stimulatory molecule. In some embodiments, the CAR comprises a chimeric fusion protein comprising an extracellular antigen binding domain specific for ROR2, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain specific for ROR2, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain specific for ROR2, a transmembrane domain and an intracellular signaling domain comprising at least two functional signaling domains derived from one or more costimulatory molecule(s) and a functional signaling domain derived from a stimulatory molecule.

In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

In particular aspects, CARs comprise fusions of single-chain variable fragments (scFv) derived from monoclonal antibodies that are specific for ROR2, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In some embodiments, the chimeric antigen receptor of the present invention comprises at least one VH and/or VL sequence of an antibody that is specific for ROR2. In some embodiments, the portion of the CAR of the invention comprising an antibody or antibody fragment thereof that is specific for ROR2 may exist in a variety of forms where the antigen binding domain is expressed as part of a contiguous polypeptide chain including, for example, a single domain antibody fragment(sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment specific for ROR2. In a further aspect, the CAR comprises an antibody fragment that comprises a scFv that is specific for ROR2.

Methods for preparing CAR cells are well known in the art. In some embodiments, the cell (e.g., T cell) is transduced with a viral vector encoding a CAR. In some embodiments, the viral vector is a retroviral vector. In some embodiments, the viral vector is a lentiviral vector. In some embodiments, the cell may stably express the CAR. In some embodiments, the cell (e.g., T cell) is transfected with a nucleic acid, e.g., mRNA, cDNA, DNA, encoding a CAR. In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

In some embodiments, the CAR activity can be controlled if desirable to optimize the safety and efficacy of a CAR therapy. There are many ways CAR activities can be regulated. For example, inducible apoptosis using, e.g., a caspase fused to a dimerization domain (see, e.g., Di et al., N Egnl. J. Med. 2011 Nov. 3; 365 (18): 1673-1683), can be used as a safety switch in the CAR therapy of the instant invention.

### Pharmaceutical Compositions

Typically, the agent of the present invention is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium s carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. ROR2 is highly expressed in patient cells. (A) ROR2 expression (western blot) in ALCL cell lines (ALKIma (CRISPR engineered cell lines), SUDHL1 (patient ALCL cell line), NA-OE (NPM-ALK overexpression) and PDX (Patient established cell line))(control: activated

T lymphocytes). (B) ROR2 expression (RNAseq data) in CRISPR/Cas9 engineered translocated and mice tumors cells. (C) ROR2 expression (RNAseq data) in ALCL ALK(+) patient tumor samples showing high expression of this receptor in all samples (compared to control reactive lymphnodes). (D) ROR2 expression is correlated to NPM-ALK(+) expression in ALCL ALK(+) patient tumor samples. (E) ROR2 expression is upregulated in crizotinib resistant clones (CRISPR engineered cell lines)

FIG. 2. The ROR2 antibody specifically recognizes patient derived ALCL cell lines (Karpas, Pio and SUPM2 cell lines) compared to negative control cell lines (T leukemia cell line, Jurkat)

FIG. 3. ROR2 is expressed in almost all ALK+ ALCL patient samples and some ALK-ALCL patient samples: ROR2 expression reanalysed from published expression data (GSE65823 data) for ALK+ and ALK-ALCL patients

### EXAMPLE

### Methods

### Primary Cells and Cell Lines

PBMCs were isolated using SepMate™-50 (IVD)(Stem-Cell Technologies #85450) following manufacturer's instructions. PBMCs were activated 5 to 7 days on coated plates with anti-CD3-OKT3 (Biolegend #317325 RRID: AB_11147370) and 1 ng/uL anti-CD28 (eBioscience #16-0289-81 RRID: AB_468926) in RPMI medium (Invitrogen) supplemented with 20% heat-inactivated Fetal Bovin Serum (GIBCO). After activation cells were directly transfected with the RiboNucleoProtein RNP/Cas9 complex. ALK+ ALCL cell lines and patient-derived xenograft(PDX) were cultivated in RPMI (Invitrogen) medium supplemented with 20% heat inactivated Fetal Bovine Serum (GIBCO).

### CRISPR/Cas9 Transfection and Translocation Frequency

T lymphocytes were transfected at 5 to 7 days post CD3/CD28 activation, with the RNP/Cas9 complex using the 4D Nucleofector Amaxa technology (Lonza)(using the gRNA targeting NPM and gRNA targeting ALK and the Cas9 protein (quantity ratio 2:1). IL-2 (40 U/mL) was added in the media once at the time of transfection but never used afterwards. Transfected cells were long term maintained in 20% heat-inactivated FBS complemented RPMI medium.

### RNA-seq and Bioinformatics Analysis

Sequencing was carried out using 2×100 cycles (pairedend reads, 100 nucleotides) for all samples on the Illumina NovaSeq6000 instrument. Reads were quantified with salmon v0.14.1(genome GRCh38) and differential analysis was performed using the R package DESeq2 (R version 4.0.3 and DESeq2 version 1.30.1). No statistical methods were used to predetermine sample size. RNAseq experiments were performed in triplicates. All GSEA analyses (version 4.1.0) were performed using the pre-ranked mode because of the weak number of samples for each condition in data coming from the model. In order to identify genes harboring a strong progressive up-regulation in the model's dataset, genes that harbored an overexpression associated to a LFC2 higher than 2 (and obligatorily associated with an adjusted p-value lower than 5%) both between conditions WT (wild type) and NPM-ALK in vitro as well as between conditions NPM-ALK in vitro and NPM-ALK in vivo were selected.

### Animal Experiments

NSG immunodeficient mice (NOD.Cg-Prkdc (scid) Il2rg(tm1Wj1)/SzJ (the Jackson Laboratory, Bar Harbor, ME, USA) were maintained at the Gustave Roussy preclinical facility and NOD/SCID Gamma (NSG NOD-prkdcscid) mice (Janvier Labs) for subcutaneous experiments were housed at the CRCT facility. For xenograft tumor assay, a total of 3×10⁶ ALKImal cells were injected subcutaneously into both flanks of 5-weekold female NSG mice. For intravenous injections, 8 to 12-weeks old NSG mice were irradiated at 1.5 Gy, and 0.7 to 3 million human cells were injected intravenously (i.v.). Disease progression was monitored by flow cytometry of mouse peripheral blood drawn periodically by submandibular bleeds. Mice were sacrificed when engraftment reached at least 30% or upon reaching a defined disease endpoint.

### Histological Analysis

Subcutaneous tumors or organs were excised and sections were fixed in 10% neutral buffered formalin and embedded in paraffin for staining with H&E. For histological analyses, sample organs were stained with hematoxylin and eosin. Briefly, the slides were heattreated for antigen retrieval using CC1 buffer (pH 8) and incubated with pre-diluted primary antibodies to anti-ALK1 (clone ALK-01), anti-CD30 (clone Ber-H2), anti-CD4 (clone SP35) and anti-CD3 (clone 2GV6)(all from Ventana, Roche Diagnostics) and anti-ROR2 (Abcam #ab218105). Epitopes were subsequently visualized using the Opti View DAB detection method (Ventana, Roche Diagnostics) and nuclei were counterstained with haematoxylin. For interpretation, the slides were evaluated by light microscopy.

### Results

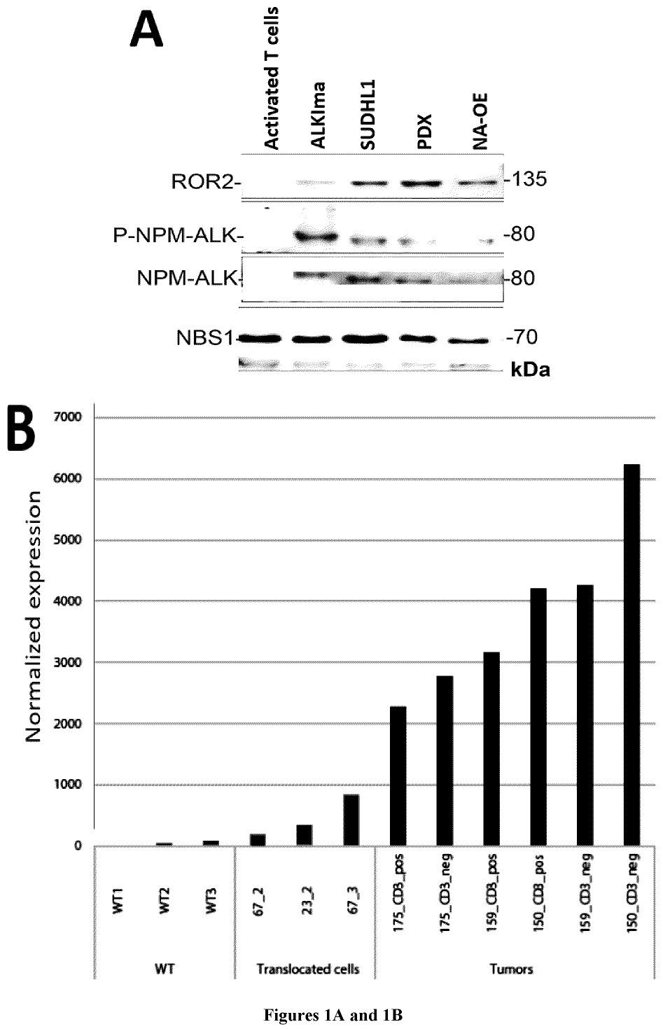

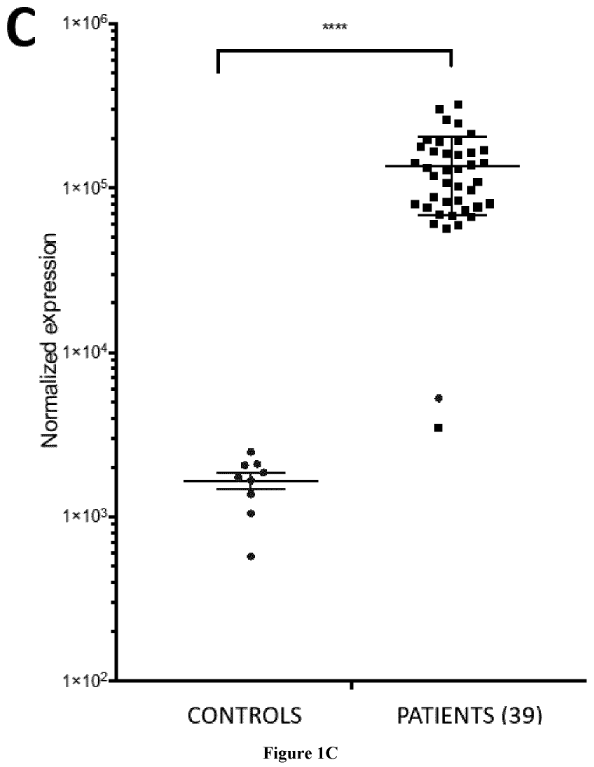

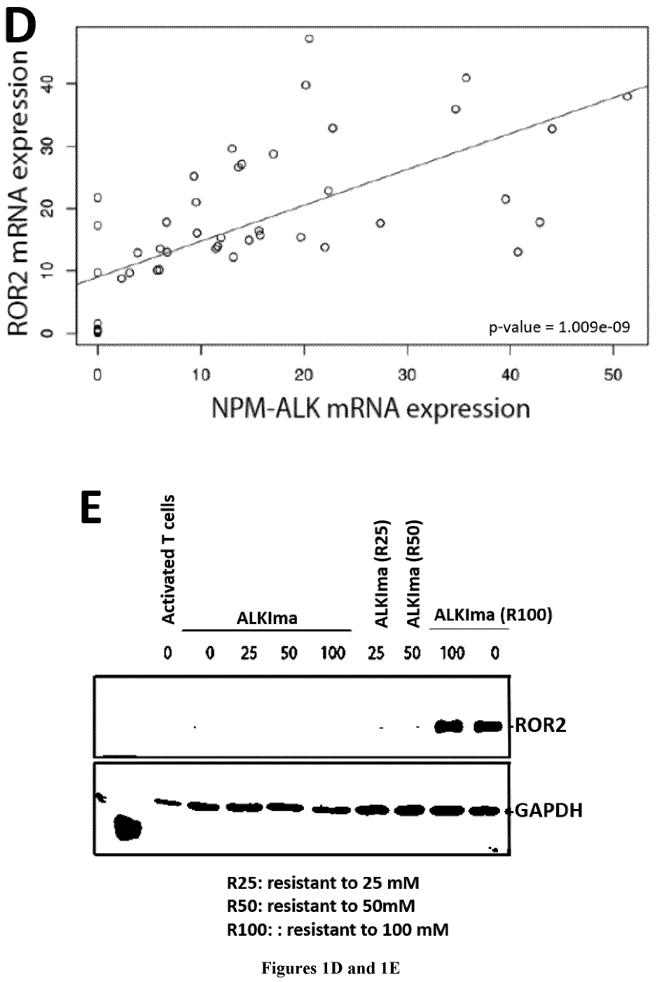

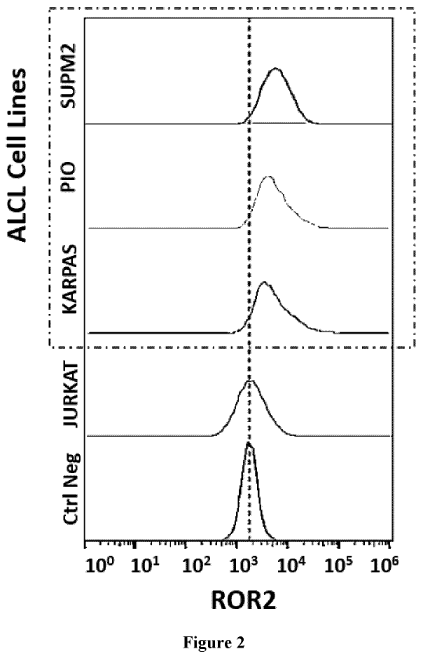

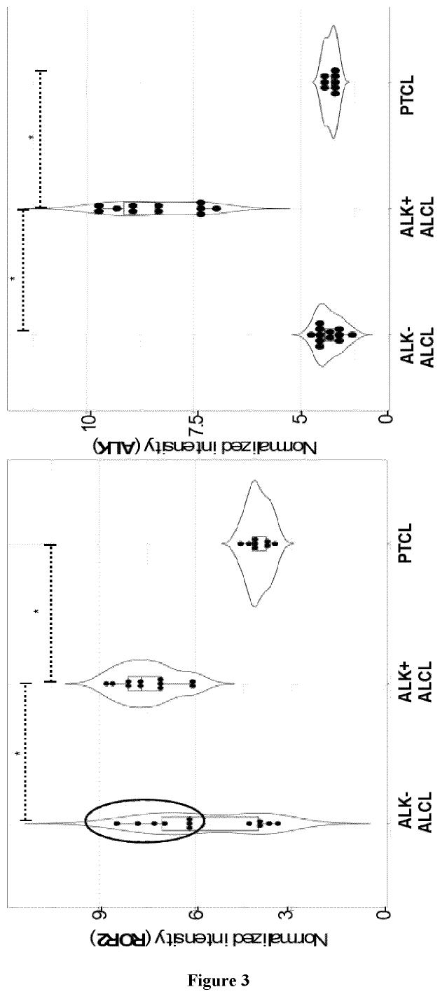

Anaplastic large cell lymphoma (ALCL) is a rare and aggressive peripheral T-cell lymphoma affects lymph nodes and extra-nodal sites with characteristic skin lesions. Approximatively half of the tumors express the NPMI-ALK fusion from the translocation t(2;5)(p23;q32). In the present study, we demonstrate high-efficacy transformation of primary human (mature) T-cells upon precise engineering of the t(2;5)(p23;q35) translocation. Our data show that human T cell survival increases drastically upon NPMI-ALK translocation induction, up to several months in cytokine free medium while normal T cells die in a few weeks. Immunodeficient mice transplanted with the NPM1-ALK(+) cells developed systemic disease in few months following injections, with nodal and skin involvements strikingly resembling human disease features. Interestingly we observe the tumor formation with various types of cells recapitulating both the immune phenotype diversity (including the presence of CD8+ tumor cells), and the histological pattern (large or small cells) of NPM1-ALK(+) ALCL patient cells. Transcriptomic signature of engineered translocated clones in vivo (tumors) perfectly recapitulate patient tumors. Using progression analysis (from mature T cells to in vivo tumors including pre transformed in vitro clones), we identify ROR2 as progressively up regulated thought tumorigenesis. ROR2 is a transmembrane receptor expressed at high level during early development and at low level in adult tissue. This receptor has been implicated in Wnt signaling pathway. Patient samples show a significantly high ROR2 expression (transcriptomic data) as well as a strong ROR2 protein expression (IHC) with some tumors displaying a clear membrane signal (25/27). ROR2 is expressed in almost all ALK+ ALCL patient samples and some ALK− ALCL patient samples (FIG. 3). More precisely we found positive expression of ROR2 also in half of the ALK− ALCL samples. Noticeably all ALK+ ALCL samples resistant to chemotherapies were ROR2 positive. ROR2 mRNA expression level is also positively correlated to NPM-ALK expression level in tumor cells and is not expressed in normal T cells. In addition, ROR2 protein level is significantly increased in resistant cells to the ALK inhibitor, crizotinib, used in clinical trials for children with refractory tumors (FIGS. 1A to 1E). ROR2 antibody specifically recognizes patient derived ALCL cell lines (FIG. 2)

This result opens the road to ROR2 specific therapies: ROR2 inhibitors, monoclonal antibodies therapies or even ROR2 specific CAR-T cells, including for ALCL ALK(+) resistant tumors.

### REFERENCES

Throughout this application, various references describe the state of the art to which this invention pertains. The disclosures of these references are hereby incorporated by reference into the present disclosure.

1. A method of treating an anaplastic large cell lymphoma (ALCL) in patient in need thereof comprising administering to the patient a therapeutically effective amount of an agent capable of inducing cell death of ROR2 expressing cancer cells.

2. The method of claim 1, wherein the (ALCL) is resistant to chemotherapy.

3. The method of claim 1 wherein the anaplastic large cell lymphoma is ALK positive.

4. The method of claim 1, wherein the anaplastic large cell lymphoma is ALK negative.

5. A method of treating an ALK positive anaplastic large cell lymphoma in a patient in need thereof and/or enhancing the potency of a ALK inhibitor administered to the patient suffering from the ALK positive anaplastic large cell lymphoma as part of a treatment regimen comprising administering to the patient a therapeutically effective combination comprising at least one ALK inhibitor and an agent capable of inducing cell death of ROR2 expressing cancer cells.

6. The method of claim 5, wherein the ALK positive anaplastic large cell lymphoma is resistant to chemotherapy.

7. The method of claim 6, wherein the ALK positive anaplastic large cell lymphoma is resistant to ALK inhibitors.

8. (canceled)

9. A method of preventing resistance to chemotherapy in a patient suffering from a cancer comprising administering to the patient a therapeutically effective amount of an agent capable of inducing cell death of ROR2 expressing cancer cells.

10. The method of of claim 9, wherein the resistance is resistance to an ALK inhibitor.

11. The method of claim 5, wherein the ALK inhibitor is selected from the group consisting of crizotinib, alectinib, TAE684, CEP28122, and Lorlatinib.

12. The method of claim 1 wherein the agent is ROR2 inhibitor.

13. The method of claim 1 wherein the agent is an antibody having binding affinity for ROR2.

14. The method of claim 13 wherein the agent is an antibody directed against at least one extracellular domain of ROR2 and leads to the depletion of ROR2 expression cancer cells.

15. The method of claim 14 wherein the antibody mediates antibody-dependent cell-mediated cytotoxicity.

16. The method of claim 14 wherein the antibody is a multispecific antibody comprising a first antigen binding site directed against ROR2 and at least one second antigen binding site directed against an effector cell.

17. The method of claim 14 wherein the antibody is conjugated to a cytotoxic moiety.

18. The method of claim 1 wherein the agent is a CAR cell wherein the CAR comprises at least an extracellular antigen binding domain specific for ROR2.

19. The method of claim 18 wherein the CAR cell is a CAR-T cell, a CAR-NK cell or a CAR-MAIT cell.

20. The method of claim 2, wherein the chemotherapy is a combination a cyclophosphamide, doxorubicin, vincristine and prednisone (CHOP).


SEQUENCE LISTING (2 sequences)

SEQ ID NO: 1 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 2 - protein, 943 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA